(19) United States
(12) Patent Application Publication
(10) Pub. No.: US 2016/0324752 A1
(43) Pub. Date: Nov. 10, 2016

(54) ASCORBIC ACID AND BIOPOLYMER COSMETIC COMPOSITIONS

(71) Applicant: Makefield LLC, Newtown, PA (US)

(72) Inventors: Kalyan Vepuri, Newtown, PA (US); Irwin Palefsky, Fairfield, NJ (US); Russell Grandis, Paramus, NJ (US)

(21) Appl. No.: 15/146,480

(22) Filed: May 4, 2016

Related U.S. Application Data
(60) Provisional application No. 62/156,650, filed on May 4, 2015.

(51) Int. Cl. A61K 8/67; A61K 8/58; A61K 8/891; A61K 8/37; A61Q 1/02; A61K 8/29; A61K 8/43; A61K 8/25; A61K 8/49; A61K 8/60; A61K 8/81; A61K 8/73; A61Q 19/08

(52) U.S. Cl. CPC A61K 8/676; A61K 8/735; A61K 8/585; A61K 8/891; A61K 8/37; A61Q 1/02; A61Q 19/08; A61K 8/43; A61K 8/25; A61K 8/498; A61K 8/606; A61K 8/8152; A61K 8/29; A61K 2800/43; A61K 2800/522; A61K 2800/49

(57) ABSTRACT

Cosmetic compositions and methods of treating aspects of the skin associated with aging and/or photo-aging are disclosed herein.

### CROSS-REFERENCE TO RELATED APPLICATION

The present application claims priority to U.S. Application No. 62/156,650, filed on May 4, 2015, by Kalyan Vepuri, Lauren Hoffman, Irwin Palessky and Russ Gandis, the disclosure of which is incorporated herein by reference in its entirety.

### FIELD OF THE INVENTION

The invention generally relates to cosmetic compositions for alleviating signs of aging of the skin and methods of use thereof.

### BACKGROUND OF THE INVENTION

The skin constitutes a barrier against external attacks. The quality of the skin barrier and of the mucous membranes is affected daily by physiological or external stresses. Such stresses cause, at the level of the skin, numerous modifications and degradations which result in irritation reactions, or even inflammatory reactions. Moreover, these inflammatory and irritation reactions may be accompanied by drying of the skin and are more widely involved in the overall modification of skin homeostasis.

In the course of aging of the skin, changes in the structure and function of the skin become apparent. One of these main signs is the appearance of fine lines and deep wrinkles, the size and the number of which increase with age.

It is common practice to treat these conditions with cosmetic compositions containing active agents such as α-hydroxy acids, β-hydroxy acids, ascorbic acid and retinoids. These agents act on wrinkles by decreasing the dead cells of the skin and accelerating the process of cell renewal and/or by increasing the synthesis of extracellular matrix components or preventing degradation. However, these active agents have the drawback of being effective in treating wrinkles only after they have been applied for a certain amount of time, ranging from a few days to several weeks.

There is therefore a need for compositions producing an immediate effect, leading rapidly to smoothing-out of the wrinkles and fine lines and to the disappearance, even temporary, of fatigue marks. Such compositions contain tensioning agents. These tensioning agents may be polymers of natural or synthetic origin, such as polyhydroxyacids, capable of forming a film that causes shrinkage of the stratum corneum, the superficial layer of the epidermis.

However, the efficacy of current tensioning agents is limited over time, and tends to disappear in the course of a day. Such tensioning agents are effective only on the surface wrinkles of the skin, and have less effect on the deeper wrinkles, for which their action is poor and short-lived. In addition, the tensioning effect that they afford does not last very long, since the film formed on the skin has a tendency to crack as a result of facial expressions. One reason for this transient effect is that these tensioning agents form a relatively rigid and inflexible film on the skin.

It is therefore an object of the present invention to provide a composition providing rapid relief for the signs of aging of the skin, including fine lines and deep wrinkles.

It is a further object of the present invention to provide a method of reducing or treating unaesthetic aspects of the skin associated with aging or photo-aging.

### SUMMARY OF THE INVENTION

Compositions and methods to reduce the appearance of fine lines and wrinkles caused by aging or photo-aging while improving the elasticity and moisturization of dry and damaged skin are described herein. An exemplary composition contains a combination of L-ascorbic acid and a biopolymer containing hyaluronic acid and glucommannan. L-ascorbic acid treats the signs of aging by accelerating the process of cell renewal and builds collagen. In one embodiment, L-ascorbic acid is in an amount of 8.0-12% by weight of the composition, and preferably in an amount of 10% by weight of the composition. The biopolymer possesses hygroscopic properties and acts by plumping the skin from the inside out, smoothing out the wrinkles and fine lines at the surface of the skin. In one embodiment, the biopolymer is hyaluronic acid crosslinked with glucommannan.

In preferred embodiments, the composition contains dermatological agents, occlusive agents, emollients, emulsifiers and pigments. In one embodiment, the dermatological agent is an antioxidant. Preferred antioxidants include retinol palmitate, panthenyl triacetate, and tocopheryl acetate, and combinations thereof. The antioxidants act by inhibiting free radical damage caused by aging or environmental damage to the skin. In another embodiment, the dermatological agent is adenosine, dimethylmethoxy chromanol, creatine, or combinations thereof. In one embodiment, the occlusive agent is ethylhexyl hydroxystearate. Occlusive agents act by forming a barrier over the skin. This barrier reduces the evaporation of water from the skin, leading to increased moisture in the epidermis and enhancing the delivery of the agents. In another embodiment, the composition contains L-ascorbic acid, ethylhexyl hydoxystearate, cyclopentasiloxane and polysilicone-11 in an amount of between 40 and 60% by weight of the composition, and preferably in an amount of 50% by weight of the composition. Emollients are an externally applied compound that softens or soothes skin. In one embodiment, the emollient is dimethicone. The composition can contain one or more emulsifiers. Emulsifiers are surface active substances which promote the suspension of one liquid in another and promote the formation of a stable mixture, or emulsion, of oil and water. In preferred embodiments, the emulsifier is a combination of cetyl PEG and PPG-10. The composition can contain one or more pigments. In preferred embodiments, the pigment is a combination of mica, titanium dioxide and carmine.

The composition can be formulated as a cream, lotion or solution.

In certain embodiments, the composition is applied between one and three times a day, preferably once a day.

### BRIEF DESCRIPTION OF THE DRAWING

FIG. 1 is a bar graph showing the differences in roughness reduction [SEr] of 30 subjects treated with Composition I over several time points.

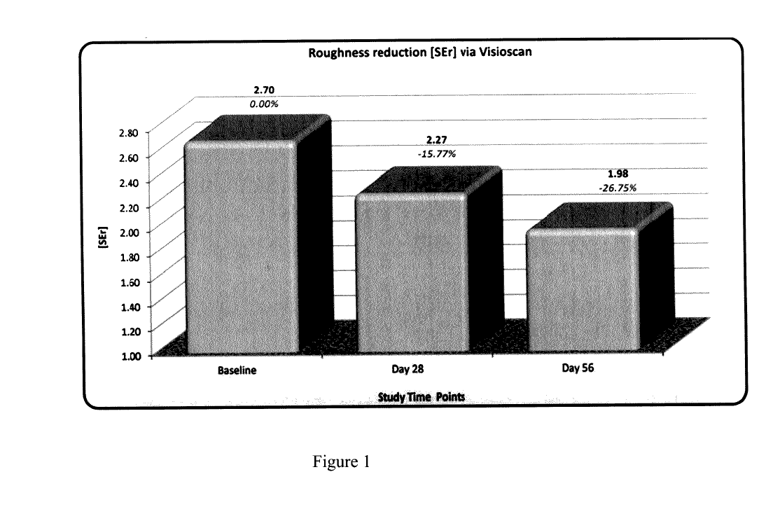

### DETAILED DESCRIPTION OF THE INVENTION

### I. Definitions

“Skin” is used herein to refer to an organ containing the epidermis, the dermis and a deep compartment, which is the hypodermis. The phrase “signs of aging of the skin” means any change in the outer appearance of the skin or in its texture due to chronological or photo-induced aging, for instance wrinkles, fine lines, wizened skin, flaccid skin, thinned skin and lack of elasticity or tonus of the skin.

An “effective amount” of the active agents described herein, refers to an amount of the active agents which, when applied, reduces or treats signs of aging of the skin.

“Cosmetically acceptable” refers to those compounds, materials, compositions, or dosage forms which are, within the scope of sound medical judgment, suitable for use in contact with the tissues of human beings without excessive toxicity, irritation, allergic response, or other problems or complications commensurate with a reasonable benefit/risk ratio.

“Pharmaceutically acceptable” refers to a material, compound, or composition which is suitable for use in contact with the skin

“Derivative” is used herein to a compound that possesses the same core as the parent compound, but differs from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical processes.

The term “crosslinking agent”, as used herein, refers to a compound capable of crosslinking hyaluronic acid with glucomannan. Exemplary crosslinking agents include, but are not limited to, terephthalic acid dichloride, phthalic acid dichloride, sebacic acid dichloride, succinic acid dichloride, fumaric acid dichloride, glutaric acid, oxalic acid dichloride, malonic acid dichloride, adipic acid dichloride, pimelic dichloride, azelaic acid dichloride, dodecanoic acid, undecanoic acid dichloride, a trichloride of a tricarboxylic acid such as citric acid, and an acid dianhydride such as succinic dianhydride.

### II. Compositions

A. Active Agents
Ascorbic acid and Derivatives

Ascorbic acid includes all enantiomers whether singly or in combination. In a preferred embodiment, the ascorbic acid is L-ascorbic acid. Ascorbic acid derivatives include salts and esters of ascorbic acid.

Salts include, but are not limited to, alkaline earth metals such as calcium, magnesium, and sodium and from the phosphates and sulfates. The ascorbic acid phosphate can be L-ascorbic acid 3-phosphate, L-ascorbic acid 2-phosphate, L-ascorbic acid 3-pyrophosphate and bis L-ascorbic acid 3,3-) phosphate. In some embodiments, the ascorbic acid phosphate is magnesium or sodium ascorbyl phosphate, and preferably, magnesium ascorbyl phosphate. Likewise, the ascorbic acid sulfate salt can be L-ascorbic acid 3-sulfate, L-ascorbic acid 2-sulfate, L-ascorbic acid 3-pyrosulfate and bis(L-ascorbic acid 3,3-)sulfate.

Ascorbic acid esters may be selected from the group consisting of fatty acid mono, di, tri, or tetra-esters of ascorbic acid. Non-limiting examples are ascorbyl palmitate, ascorbyl laureate, ascorbyl myristate, ascorbyl stearate, ascorbyl dipalmitate, ascorbyl dilaurate, ascorbyl dimyristate, ascorbyl distearate, ascorbyl tripalmitate, ascorbyl trilaurate, ascorbyl trimyristate, ascorbyl tristearate, ascorbyl tetrapalmitate (tetrahexyldecyl ascorbate), ascorbyl tetralaurate, ascorbyl tetramyristate and ascorbyl tetrastearate.
ii. Hyaluronic Acid

Hyaluronic acid is a naturally occurring, water soluble polysaccharide, specifically a glycosaminoglycan, which is a component of the extra-cellular matrix. This polysaccharide is widely distributed in human tissues. Hyaluronic acid has the ability to bind to large amounts of water, making it an excellent volumizer of soft tissues. Hyaluronic acid has a weight-average molecular weight ranging from 50 to 3,000 kDa, preferably ranging from 50 to 2500 kDa and preferentially ranging from 500 to 2,000kDa. As illustrations of different hyaluronic acid fractions, reference is made to Stern, R. et al., “Hyaluronan fragments: An Information-Rich System”, European Journal of Cell Biology, 2006, 58, 699-715. This article provides biological activities of hyaluronic acid as a function of its molecular weight.

Salts of hyaluronic acid are pharmaceutically acceptable, preferably dermatologically acceptable. The salts of hyaluronic acid are selected from the hydrolyzed calcium hyaluronate, hydrolyzed sodium hyaluronate, calcium hyaluronate, potassium hyaluronate, sodium hyaluronate, sodium hyaluronate and sulfated mixtures. All such forms of hyaluronic acid are encompassed.

Hyaluronic acid may be provided by the company Hyactive under the trade name CPN (MW: 10 to 150 kDa), by the company Soliance under the trade name Cristalhyal (MW: 1.1x1O6), by the company Bioland under the name Nutra HA (MW: 820 kDa), by the company Bioland under the name Nutra AF (MW: 69 ka), by the company Bioland under the name Oligo HA (MW: 6100 Da) or by the company Vam Farma Cosmetica under the name D Factor (MW: 380 Da), by the company LCA Pharmaceutical under the trade name Hyaluderm, by the company Corneal under the trade name Juvelift Corneal, by the company Q-Med under the trade name Restylane Touch Line, or by the company Revitacare under the trade name Revitacare Biorevitalization.
iii. Glucomannan

Glucomannan is a polysaccharide that is derived from konjac root (Amorphophallus konjac), Amorphophallus konjac is a perennial herbaceous herb. it has been used as a food and food additive in China and Japan for more than 1000 years. The fresh konjac tuber contains an average of 13% dry matter. Sixty four percent of the dry matter is glucomannan and 30% is starch. The molecular mass of glucomannan derived from the konjac tuber is 200 to 2,000 kDa, depending upon konjac species or variety and processing method. Glucomannan can absorb up to 200 times its weight in water. Glucomannan derived from the konjac root is a high molecular weight polysaccharide formed from residues of glucose (G) and mannose (M) in a proportion of 5:8 bound together by β-1,4-linkages.

Glucomannan is not a linear molecule and has short side chains of 11-16 monosaccharides occurring at intervals of 50-60 units of the main chain attached by 1 β→3 linkages. Acetyl groups on carbon 6 are located on every 9-19th sugar unit of the main chain. These acetyl groups contribute to the solubility and gelling properties. Glucomannans from other sources may contain glucose and mannose in different proportions and/or different sequences, as well as minor amounts of sugars other than glucose and mannose. All such forms of glucomannan are encompassed.
iv. Dermatological Agents

The composition can also contain dermatological agents for improving the appearance and comfort of the skin. The dermatological agent can be an antioxidant. Examples of antioxidants include, but are not limited to, tocopheryls, retinoids, BHT, camellia sinensis leaf extract, carotenoids, resveratrol, triethyl citrate, arbutin, superoxide dismutase, zinc, sodium metabisulfite, lycopene, and ubiquinone or combinations thereof. Preferred antioxidants include retinol palmitate, panthenyl triacetate, and tocopheryl acetate. Additional examples of dermatological agents are dimethylmethoxy chromanol, creatine and adensoine.

B. Excipients

The composition can contain one or more cosmetically acceptable excipients. Cosmetically acceptable excipients include, but are not limited to, water, preservatives, chelating agents, sunscreen agents, vitamins, dyes, pigments proteins, amino acids, natural extracts such as plant extracts, humectants, fragrances, perfumes, oils, emollients, lubricants, butters, penetrants, thickeners, viscosity modifiers, polymers, resins, film formers, surfactants, detergents, emulsifiers, opacifying agents, volatiles, propellants, liquid vehicles, carriers, salts, pH adjusting agents (e.g., citric acid), neutralizing agents, buffers, absorbents, and combinations thereof.
i. Occlusive Agents

Occlusive agents provide a hydration barrier to the skin. An occlusive layer or hydration barrier is a layer or barrier sufficient to result in reduction in epidermal water loss, which results in skin hydration. Suitable occlusive agents include, but are not limited to, mineral oils and greases, long chain acids, animal fats and greases, vegetable fats and greases, and water insoluble polymers. In a preferred embodiment, the occlusive agent is ethylhexyl hydroxystearate.
ii. Emollients

Emollients protect against wetness or irritation, softens, soothes, coats, lubricates, moisturizes, protects, and/or cleanses the skin. Suitable emollients for include, but are not limited to, a silicone compound (e.g., dimethicone, cyclomethicone, dimethicone copolyol or a mixture of cyclopentasiloxane and dimethicone/vinyldimethicone cross polymer, cyclopentasiloxane polysilicone), polyols such as sorbitol, glycerin, propylene glycol, ethylene glycol, polyethylene glycol, caprylyl glycol, polypropylene glycol, 1,3-butane diol, hexylene glycol, isoprene glycol, xylitol, ethylhexyl palmitate, a triglyceride such as caprylic/capric triglyceride and fatty acid ester such as cetearyl isononanoate or cetyl palmitate. More than one emollient may be included in the formulation. In a specific embodiment, the emollient is dimethicone, creatine, amidodimethicone, dimethiconol, cyclopentasiloxane, potassium dimethicone PEG-7 panthenyl phosphate, or a combination thereof.
iii. Emulsifiers

The compositions can contain one or more emulsifiers. Suitable emulsifiers include, but are not limited to, copolymers of an unsaturated ester and styrene sulfonate monomer, cetearyl alcohol, glyceryl ester, polyoxyethylene glycol ether of cetearyl alcohol, stearic acid, polysorbate-20, ceteareth-20, lecithin, glycol stearate, polysorbate-60, or polysorbate-80, or combinations thereof. More than one emulsifier may be included in the formulation. In preferred embodiments, the emulsifier is a combination of cetyl PEG and PPG-10.
iv. Preservatives

One or more preservatives may be included in the composition to prevent microbial growth. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), potassium sorbate, and/or grapefruit seed extract, or combinations thereof. More than one preservative may be included in the formulation. Other preservatives are known in the cosmetics industries and include salicylic acid, DMDM hydantoin, formaldahyde, chlorphenism, triclosan, imidazolidinyl urea, diazolidinyl urea, sorbic acid, methylisothiazolinone, sodium dehydroacetate, dehydroacetic acid, quaternium-15, stearalkonium chloride, zinc pyrithione, sodium metabisulfite, 2-bromo-2-nitropropane, chlorhexidine digluconate, polyaminopropyl biguanide, benzalkonium chloride, sodium sulfite, sodium salicylate, citric acid, neem oil, essential oils, lactic acid, and vitamin e (tocopherol).
v. Pigments

Pigments are used as coloring agents that provide color to cosmetic compositions. Pigments include, but are not limited to, titanium dioxide, optionally surface-treated, zirconium oxide or cerium oxide, and also zinc oxide, iron (black, yellow or red) oxide or chromium oxide, manganese violet, ultramarine blue, chromium hydrate and ferric blue, metal powders (e.g. aluminium powder and copper powder), cochineal carmine, organic pigments of azo dyes, anthraquinone dyes, indigoid dyes, xanthene dyes, pyrene dyes, quinoline dyes, triphenylmethane dyes and fluoran dyes.

### III. Methods of Manufacture

The composition can be in various galenical forms conventionally used for topical applications and in particular in the form of dispersions of the lotion or serum type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or wax/aqueous phase dispersions. These compositions are prepared according to the well kwon methods to those skilled in the art.

In addition, the compositions may be more or less fluid and may have the appearance of a gel, a white or colored cream, an ointment, a milk, a lotion, a serum, a paste or a mousse.

In one preferred embodiment, the composition is in the form of an oil-in-water emulsion. The composition may also be in the form of an emulsified gel. In another embodiment, the composition is in the form of an O/W emulsion. In yet another embodiment, the composition is in the form of an W/O emulsion.

The composition preferably has a skin-friendly pH, generally in the range between 4 and 8, and more preferably between 4.5 and 6.5.

### IV. Methods of Use

The compositions are used for cosmetic treatment of the skin, including the scalp, and the mucous membranes (lips). The compositions can be used for reducing the visible or tactile irregularities of the surface of the skin, in particular for reducing wrinkles and fine lines and/or blemishes on the skin and/or smoothing and/or firming the skin and/or unifying the complexion.

The compositions also find application in attenuating surface irregularities of the skin and in improving the skin's microrelief, in particular attenuating actinic lentigo and acne or chickenpox scars, and unblocking skin pores; and treating dry skin and acne-prone greasy skin. With respect to greasy skin, this is often associated with a desquamation defect, and with a thick skin grain. Furthermore, the excess sebum may serve as a support for the anarchic growth of saprophytic bacterial flora (in particular Propionibacterium acnes and Pityrosporum ovale), and cause the appearance of comedones and/or acne scars. These acne scars are another cutaneous sign of greasy skin that may advantageously be combated by means of the use the composition.

### EXAMPLES

### Example 1: Manufacture of Composition 1

### Materials and Methods

TABLE 1
Cosmetic Composition Phase A

| | | | Batch |
|---|---|---|---|
| Ingredient | | | Weight |
| (Tradename) | INCI Designation | Supplier | (g) |
| GRANACTIVE ® | Cyclopentasiloxane | Grant | 500 |
| AA-20 | Ascorbic Acid | | |
| | Polysilicone-11 | | |
| | Ethylhexyl | | |
| | Hydroxystearate | | |
| XIAMETER ® | Dimethicone | Corning/Xiameter/ | 69.3 |
| PMX-200 Slicone | | Univar | |
| Fluid 100 CST | | | |
| GRANSIL ® Eps | Polysilicone-11 | Rant | 20 |
| | Laureth-12 | | |
| BENTONE ® Gel | Cyclopentasiloxane | Elementis/Essential | 160 |
| Vs-5 Pc V Hv | Disteardimonium | Ingredients | |
| | Hectorite | | |
| | Propylene Carbonate | | |

The ingredients in Table 1 were mixed until uniform. The combination was then homogenized for 10 minutes at 3000 RPM to afford Phase A.

TABLE 2
Cosmetic Composition Phase B/C

| Ingredient | | | |
|---|---|---|---|
| (Tradename) | INCI Designation | Supplier | Batch Size |
| LIPOCHROMAN ® | Dimethylmethoxy | Lipotec | 0.5 |
| Es290 | Chromanol | | |
| TEGO COSMO ® C | Creatine | Evonik/Upi | 5.0 |
| 100 | | | |
| ABIL EM ® 90 | Cetyl PEG/PPG-10/1 | Evonik/Upi | 40 |
| | Dimethicone | | |

Dimethylmethoxy chromanol and creatine were added to a mixture of cetyl PEG, PPG-10 and 1-dimethicone to afford Phase B/C. Phase B/C was heated to 40° C. until all the powders dissolved.

TABLE 3
Cosmetic Composition Phase B/C/D

| Ingredient | | | |
|---|---|---|---|
| (Tradename) | INCI Designation | Supplier | Batch Size |
| Vitamin A-Palmitate | Retinyl Palmitate | BASF/Dewolf | 5.0 |
| Care (Stabilized With | Suflower Seed Oil | | |
| 1.0 Tocopherol) | | | |
| Material No. 50131672 | | | |
| D-Panthenyltriacetate | Panthenyl Triacetate | Induchem | 30 |
| Vitamin E-Acetate | Tocopheryl acetate | BASF/Dewolf | 10 |
| Care | | | |
| Ultra Filling Spheres | Ethylhexyl Palmitate | BASF | 20 |
| C00487 | Trihydroxystearin | | |
| | Sodium Hyaluronate | | |
| | Amorphophallus | | |
| | Konjac Root Powder | | |

The ingredients in Table 3 were slowly added to Phase B/C and mixed until the mixture was uniform to afford Phase B/C/D. Phase B/C/D was added to Phase A and mixed until uniform to afford Phase E.

TABLE 4
Cosmetic Composition Phase E

| Ingredient | | | |
|---|---|---|---|
| (Tradename) | INCI Designation | Supplier | Batch Size |
| Bpa-500 | Polymethyl | Kobo | 10 |
| | Methacrylate | | |
| Adenosine #104750 | Adenosine | Specialties | 0.2 |
| Ss-500w | Silica | Kobo | 30 |
| Cloisonne Red 424c | Mica Titanium | BASF/DeWolf | 14.8 |
| | Dioxide Carmine | | |
| Flamenco Gold 220c | Mica Titanium | BASF/DeWolf | 26 |
| | Dioxide | | |
| Timica Extra Bright | Mica Titanium | BASF/DeWolf | 59.2 |
| 1500 | Dioxide | | |

Each of the ingredients in Table 4 was sprinkled into Phase E and mixed until uniform. The mixture of Phase E was homogenized for 10 minutes at 3000 RPM to afford Composition 1.

### Example 2: Clinical Trial Study

### Objective

The purpose of this study was to evaluate the performance of an anti-aging product (Composition 1) when tested over a 56 day period. Surface evaluation of living skin was conducted instrumentally using a Visioscan image analysis system.

### Standards for Inclusion

Female subjects were between 35 and 65 years old. The subjects were in general good health and free of any health problems, including neurological, dermatological, or systemic disorder that in the opinion of the Study Director would make study participation inappropriate. Individuals abstained from using any facial anti-aging products for a period of 1 week prior to the commencement of the study. Subjects abstained from use of any anti-aging products other than the assigned test articles for the duration of the study. Individuals refrained from any facial spa treatments including facials, microdermabrasion, chemical peels, Botox etc. for the duration of the study. Subjects refrained from sunbathing or tanning bed use for 7 days prior to study initiation and the entire duration of the study.

### Standards for Exclusion

Individuals under the care of a physician being treated for specific condition that may interfere with the study design at the discretion of the Study Director were excluded. Individuals currently taking medication that may mask or interfere with the test results and/or diagnosed with chronic skin allergies were excluded. The study excluded females who were pregnant, lactating, have been pregnant, or given birth within the six month period immediately preceding the commencement of the study. Subjects with a history of any form of skin cancer, melanoma, lupus, psoriasis, connective tissue disease, diabetes, or any disease that would increase the risk associated with study participation were not included. Individuals who experienced irritation or sensitivity to skin treatment products and/or known allergies or skin and/or eye conditions, which would interfere with the study at the discretion of the Study Director were excluded.

### Panel Demographics

The number of subjects enrolled in the study was thirty and thirty subjects completed the study. The race of the subjects was the following: Caucasian (27) and Hispanic (3).

### Clinical Procedure

In order to pre-condition the test sites and keep all topical treatments consistent during the study, panelists were required to abstain from using any facial anti-aging products for a period of 1 week prior to study commencement. On each evaluation day, all subjects reported with their face devoid of any topical treatments (including Composition 1),

Subjects were mandated to adhere to all the restrictions aforementioned in the inclusion/exclusion section. All participants were advised of the general nature and purpose of this study. The subjects then acclimated to the ambient environment for a period of thirty minutes prior to baseline evaluation. The same acclimation procedure was applied to 28 and 56 days evaluation time points.

The study was conducted according to the sponsor requested design wherein all subjects were instructed to choose one of the 2 shades of test product matching their skin complexion and use the test product as part of their normal daily routine according to the following sponsor supplied use following directions:
the product comes in two shades. If you have fair to medium skin tone, the pink shaded product will work best. If you have medium to a deep skin tone, you will want to use the bronze shade;
this product is to be used around your eye area one time per day and applied in the morning;
please do not use any other products around the eye when using this product;
One pump of the product should be sufficient for both eyes;
gently pat the formula into skin with your fingertips by following the outer orbital bone or what would be considered the top of your cheekbone; and

follow this outline to the temple to just below the brow to outline your eye area.

Biophysical measurements were collected at baseline and again after 28 and 56 days of daily use of the test product. In addition, all subjects completed a self-assessment questionnaire addressing consumer perception immediately after product application and again after 28 and 56 days of daily use of the test treatment. The following distinct noninvasive method was employed to establish evaluation parameters: Surface Evaluation of Living Skin (SELS)—VIOSIOCAN®.

The VISIOSCAN® (Courage-Khazaka), available for example from CK Electronic, takes a direct image of the living skin using a measuring head containing a CCD-camera and two metal halogen lamps positioned opposite each other in order to ensure even illumination of the measuring field on the skin. The grey level distribution of the pixels in the image correspond to different phenomena (white pixels represent desquamation on the skin, dark pixels represent lines and wrinkles). The software with the VISIOSCAN® automatically calculates skin roughness parameter. (Fischer, T. W., et al., “Direct and non- direct measurement techniques for analysis of skin surface topography.” Skin Pharmacol Appi Skin Physiol.1999, 12, 1-11; and Farwick, M., et al, “An EC-derived Tetrapeptide to Counterbalance ECM Degeneration” Cosmetic & Toiletries magazine, 2009, 124, 6).

### Discussion

The test product was reported by the test panelists to be effective in reducing wrinkles and improving the overall condition of the skin in the face area. The data on each of the 30 subjects are provided in Table 5.

Surface evaluation of living skin via VISIOSCAN® demonstrated decrease in (SEr) parameter associated with the depth of fine lines and wrinkles. The reduction is considered statistically significant at each evaluation time point. Table 6 describes the efficacy of the composition at various time points.

TABLE 5
Roughness reduction [SEr] via Visioscan of 30 patients

| Baseline | Day 28 | % Difference | Day 56 | % Difference |
|---|---|---|---|---|
| 2.05 | 1.43 | −30.24% | 1.56 | −23.90% |
| 1.62 | 1.60 | −1.23% | 1.13 | −30.25% |
| 3.89 | 2.87 | −26.22% | 2.26 | −41.90% |
| 4.11 | 2.66 | −35.28% | 2.36 | −42.58% |
| 2.31 | 2.23 | −3.46% | 2.16 | −6.49% |
| 2.19 | 2.09 | −4.57% | 1.35 | −38.36% |
| 2.84 | 2.17 | −23.59% | 1.73 | −39.08% |
| 2.51 | 2.20 | −12.35% | 1.94 | −22.71% |
| 1.90 | 2.00 | 5.26% | 2.03 | 6.84% |
| 2.22 | 1.58 | −28.83% | 1.76 | −20.72% |
| 2.16 | 2.14 | −0.93% | 2.05 | −5.09% |
| 3.37 | 2.92 | −13.35% | 2.58 | −23.44% |
| 4.55 | 3.99 | −12.31% | 2.98 | −34.51% |
| 3.60 | 3.13 | −13.06% | 2.40 | −33.33% |
| 2.00 | 2.02 | 1.00% | 1.93 | −3.50% |
| 4.71 | 3.13 | −33.55% | 3.12 | −33.76% |
| 2.06 | 1.94 | −5.83% | 1.46 | −29.13% |
| 1.82 | 1.60 | −12.09% | 1.80 | −1.10% |
| 1.44 | 1.54 | 6.94% | 1.39 | −3.47% |
| 1.13 | 1.20 | 6.19% | 1.19 | 5.31% |
| 3.34 | 3.39 | 1.50% | 2.49 | −25.45% |
| 2.27 | 2.08 | −8.37% | 1.83 | −19.38% |
| 2.60 | 2.13 | −18.08% | 1.75 | −32.69% |
| 1.89 | 1.35 | −28.57% | 1.73 | −8.47% |
| 1.82 | 1.66 | −8.79% | 1.52 | −16.48% |
| 1.81 | 1.60 | −11.60% | 1.45 | −19.89% |
| 5.44 | 3.35 | −38.42% | 2.92 | −46.32% |
| 2.63 | 1.98 | −24.71% | 1.61 | −38.78% |
| 3.96 | 3.31 | −16.41% | 3.06 | −22.73% |
| 2.65 | 2.84 | 7.17% | 1.71 | −35.47% |

TABLE 6
Roughness reduction [SEr] via Visioscan

| Study Time Point: | Day 28 | Day 56 |
|---|---|---|
| % Difference: | −15.77%* | −26.75%* |
| Max % Improvement: | −38.42% | −46.32% |

*Statistically Significant

Unless defined otherwise, all technical and scientific terms used herein have the same meanings as commonly understood by one of skill in the art to which the disclosed subject matter belongs. Publications cited herein and the materials for which they are cited are specifically incorporated by reference.

Those skilled in the art will recognize, or be able to ascertain using no more than routine experimentation, many equivalents to the specific embodiments described herein. Such equivalents are intended to be encompassed by the following claims. We claim:

1. A cosmetic composition comprising:
(a) an effective amount of ascorbic acid or a derivative thereof; and
(b) an effective amount of a biopolymer comprising hyaluronic acid and glucomannan, and
(c) a cosmetically acceptable excipient to topical application to the skin,
wherein the composition reduces the appearance or depth of fine lines or wrinkles.

2. The composition of claim 1, wherein the ascorbic acid is L-ascorbic acid.

3. The composition of claim 2, wherein the L-ascorbic acid is in an amount of between about 8.0 and 12% by weight of the composition.

4. The composition of claim 3, wherein the L-ascorbic acid is present in an amount of about 10% by weight of the total weight of the composition.

5. The composition of claim 4 further comprising cyclopentasiloxane, polysilicone-11, and ethylhexyl hydroxystearate.

6. The composition of claim 5, wherein L-ascorbic acid, cyclopentasiloxane, polysilicone-11, and ethylhexyl hydoxystearate are in an amount of between about 40 and about 60% by weight of the composition.

7. The composition of claim 6, wherein L-ascorbic acid, cyclopentasiloxane, polysilicone-11, and ethylhexyl hydoxystearate are in an amount of about 50% by weight of the composition.

8. The composition of claim 1, wherein the biopolymer is hyaluronic acid cross-linked with glucomannan.

9. The composition of claim 8 further comprising ethylhexyl palmitate and trihydroxystearin.

10. The composition of claim 9, wherein the biopolymer, ethylhexyl palmitate and trihydroxystearin are in an amount of between about 1.6 and about 2.4% by weight of the composition.

11. The composition of claim 10, wherein the biopolymer, ethylhexyl palmitate and trihydroxystearin are in an amount of about 2.0% by weight of the composition.

12. The composition of claim 1 further comprising dimethylmethoxy chromanol, creatine and silica.

13. The composition of claim 12, wherein dimethylmethoxy chromanol, creatine and silica are in an amount of between about 3.2 and 3.8% by weight of the composition.

14. The composition of claim 13, wherein dimethylmethoxy chromanol, creatine and silica are in an amount of about 3.55% by weight of the composition.

15. The composition of claim 1 further comprising adenosine.

16. The composition of claim 1 further comprising one or more antioxidants.

17. The composition of claim 1 further comprising one or more emulsifiers.

18. The composition of claim 1 further comprising one or more pigments.

19. The composition of claim 16 wherein the antioxidant is selected from the group consisting of retinol palmitate, panthenyl triacetate, tocopheryl acetate, and combinations thereof.

20. The composition of claim 17, wherein the emulsifier is selected from the group consisting of cetyl PEG, PPG-10 and 1-dimethicone, and combinations thereof.

21. The composition of claim 18, wherein the pigment selected from the group consisting of mica, titanium dioxide and carmine, and combinations thereof.

22. The composition of claim 1 further comprising polymethyl methacrylate.

23. The composition of claim 1 comprising:
(a) an effective amount of ascorbic acid or a derivative thereof; and
(b) an effective amount of a biopolymer comprising hyaluronic acid and glucomannan,
wherein the composition is formulated as a cream or lotion.

24. A method for reducing or treating the sign of aging of the skin comprising administering a composition comprising:
(a) an effective amount of ascorbic acid or a derivative thereof; and
(b) an effective amount of a biopolymer comprising hyaluronic acid and glucomannan.
